(19) United States
(12) Patent Application Publication
(10) Pub. No.: US 2001/0049113 A1
(43) Pub. Date: Dec. 6, 2001

(54) Diagnosis and treatment of herpes infections

(76) Inventors: Patrick W.K. Lee, Calgary (CA); Faris Farassati, Calgary (CA); An-Dao Yang, Calgary (CA)

Correspondence Address: EDWARD YOO C/O BENNETT JONES, 1000 ATCO CENTRE, 10035 - 105 STREET, EDMONTON, ALBERTA, AB, T5J3T2, CA

(21) Appl. No.: 09/681,740

(22) Filed: May 30, 2001

Related U.S. Application Data
(60) Provisional application No. 60/207,337, filed on May 30, 2000.

(51) Int. Cl. A61K 31/35; A61K 31/235; A61K 31/24; A61K 31/195; A61K 31/13; G01N 33/567; G01N 33/53; C12Q 1/02

(52) U.S. Cl. 435/7.21; 435/7.8; 435/29; 514/453; 514/533; 514/535; 514/562; 514/646; 514/665

(57) ABSTRACT

Methods and compositions for treating or preventing virus infections by interfering with the activity or function of Ras, the Ras pathway, the ERK pathway, MEK1/2, PKR or eIF-2 α, includes the use of agents which inhibit Ras or otherwise modulate anti-PKR activity. Also, a method of diagnosing such virus infections includes the use of cell lines that have an activated Ras pathway, including cell lines which have been transformed with a gene that activates the Ras pathway. The virus infections may be herpes virus infections and HSV-1 or HSV-2 infections in particular.

### Cross Reference to Related Applications

This application claims the priority benefit of U.S. Provisional Patent Application No. 60/207,337 filed on May 30, 2001.

### BACKGROUND OF INVENTION

The present invention relates to the field of treatment of viral infections and herpes virus infections in particular. The present invention also relates to the field of detection and diagnosis of the presence of herpes virus in a specimen.

The herpes family of viruses comprises viruses whose genomes consist of a single double-stranded DNA molecule. Herpes simplex viruses (HSV) are members of this family, and are known commonly for their association with cold sores (HSV-1) and genital herpes infections (HSV-2) [1]. Within this family are also varicella-zoster virus, cytomegalovirus, Epstein-Barr virus and various other human herpes viruses (HHV) such as HHV-6, HHV-7 and HHV-8 [2].

Herpes viruses, which are distributed worldwide, are generally transmitted through close personal contact. It is estimated that more than one-third of the world's population have recurrent HSV infections [1]. Upon infection, HSV has the ability to produce active disease in the host, or to enter into a latent phase, whereby the virus colonizes a host neural cell, and enters an active disease upon the appropriate stimulation. The characteristics of active herpes infection reflect virus-mediated cellular death, beginning with a ballooning of infected cells which eventually leads to cell lysis, fluid secretion, an inflammatory response, scab formation and healing [1].

Great variability exists in the symptomology of HSV infection, with asymptomatic infection being the rule, rather than the exception [1]. The severity of an active HSV infection depends upon a number of factors, especially the virus type and host immunocompetance. Most commonly known is the relatively mild infection manifesting itself as an oropharyngeal sore, commonly known as a cold sore. More severe are infections of the digits (among dental and medical personnel), eczema, disseminated cutaneous infections, and genital herpes, the lesions of which are excruciatingly painful. Most severe are neonatal infections, infections of the immunocompromised host, and encephalitis all of which can be fatal.

Herpes virus infections are usually treated with nucleoside analogs such as acyclovir, valacyclovir, famciclovir or penciclovir [1,2]. The drugs are administered either topically or systemically (orally or intravenously). Viral resistance to these drugs can occur through mutation of the target gene of the drugs which encodes the enzyme thymidine kinase. Although drug resistance is still a rare phenomenon, it has a high incidence in patients with Acquired Immune Deficiency disease (AIDs). Acyclovir-resistant HSV isolates have been identified as the cause of pneumonia, encephalitis, esophagitis and mucocutaneous infections in immunocompromised hosts [1]. Therefore, alternative drug therapies for HSV infections are required as it is anticipated that resistant forms of the virus will become more prevalent in the future. In the US, there are about 500,000 new cases of HSV infections per year [1].

Diagnosis of the virus by isolation in culture remains the definitive diagnostic test for herpes virus infection [1]. In this test, permissive cell line cultures are inoculated with what is suspected to be an infectious sample (scraping of skin vesicles, cerebrospinal fluid, stool, urine, throat, nasopharynx or conjunctivae). About 24 48 hours afterwards, the virus identity is confirmed by other tests. Various other tests have been described for the diagnosis of herpes infections, such as immunological assays, DNA assays and PCR assays. However, these suffer from disadvantages such as lack of specificity or sensitivity, lack of reproducibility, time delay, and false positives or negatives that arise because rigid controls of testing parameters are imperative.

In the case of a potential HSV infection such as encephalitis, rapid but accurate diagnosis is imperative. Inoculation of specimens onto tissue culture cells followed by immunofluorescent staining still remains the most reliable test. It would be desirable to have a mechanism of identifying herpes infections that improves upon this widely used technique. Therefore, it would be advantageous to have cell lines that allow HSV to replicate more rapidly, leading to faster and more accurate identification of HSV in the specimen.

### SUMMARY OF INVENTION

The applicants have discovered that mammalian cells that are transformed with oncogenes that activate the Ras signaling pathway are more permissive to HSV-1 infection than untransformed cells, and this permissiveness is linked to the inhibition of virus-induced activation (phosphorylation) of double-stranded RNAactivated protein kinase (PKR). By inhibiting components of the Ras pathway, the applicants were able to show that PKR phosphorylation was upregulated and viral replication was inhibited. In addition to HSV-1 and other herpes viruses, many other viruses such as reovirus, VSV or influenza viruses utilize the Ras signaling pathway. Cells which have an activated Ras pathway may be more permissive to all such viruses.

Therefore, in one aspect, the invention comprises a method of treating a viral infection, or for preventing viral infections. The method of this invention includes treating infected cells with agents that inhibit Ras pathway function or activity, agents that promote, allow or increase PKR phosphorylation, function or activity; or agents that promote, allow or increase eIF-2 α phosphorylation, function or activity. In one embodiment, this method involves the use of an agent that interferes with the function of Ras itself, such as a farnesyl-transferase inhibitor. In another embodiment, this method involves the use of an agent that interferes with the ERK pathway, and more specifically, an agent that inhibits MEK1/2 (an element downstream of ras), such as PD98059, U0124, U0125 or U0126. In one embodiment, the invention is a method of treating herpes virus infections, and HSV-1 and HSV-2 infections in particular.

In another aspect, this invention provides a pharmaceutical composition that comprises anti-viral agents that inhibit Ras pathway function or activity, agents that promote, allow or increase PKR phosphorylation, function or activity; or agents that promote, allow or increase eIF-2 α (phosphorylation, function or activity. In one embodiment, this pharmaceutical composition comprises an agent that interferes with the function of Ras itself, such as a farnesyl-transferase inhibitor. In another embodiment, this pharmaceutical composition comprises an agent that interferes with the ERK pathway, and more specifically, agents that inhibit MEK1/2, such as PD98059, U0124, U0125 or U0126.

In another aspect, the invention comprises the use of agents that inhibit Ras pathway function or activity, agents that promote, allow or increase PKR phosphorylation, function or activity; or agents that promote, allow or increase eIF-2 α phosphorylation, function or activity, for treating viral infections in a mammal or in the manufacture of a pharmaceutical compositions for treating viral infections in a mammal. In another aspect, the invention comprises a method of diagnosing virus infections in mammals using cell lines that have been transformed with a gene that activates the Ras pathway. Such method may comprise the steps of:(a) providing cultured cells which have been transfected with a gene which activates the Ras pathway; (b) inoculating the cultured cells with a specimen from the mammal with the suspected viral infection; (c) incubating the cultured cells for a length of time sufficient to allow viral protein synthesis to proceed within the cells; and (d) detecting viral proteins.

In one embodiment, the viral infection is a herpes infection and the cultured cells are murine NIH-3T3 cells that have been transformed with the oncogene Sos or v-erbB. The viral proteins may be detected by immunological techniques, such as immunofluorescence or immunoblotting.

BRIEF DESCRIPTION OF DRAWINGS

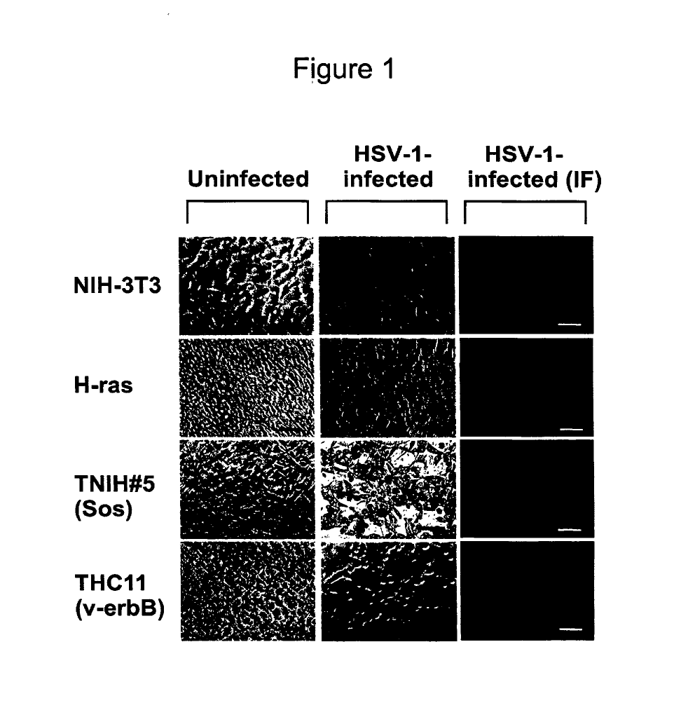

FIG. 1: A comparison, by immunoflourescence, of host cell permissiveness to HSV-1 infection of NIH-3T3 cells and NIH-3T3 cells that are transformed with various oncogenes.

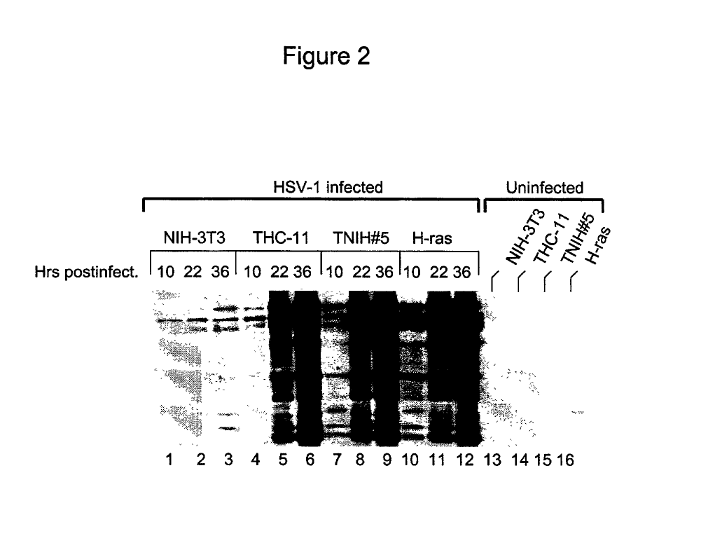

FIG. 2: A comparison, by immunoblotting of viral proteins, of permissiveness to HSV-1 infection of NIH-3T3 cells and NIH-3T3 cells that are transformed with various oncogenes.

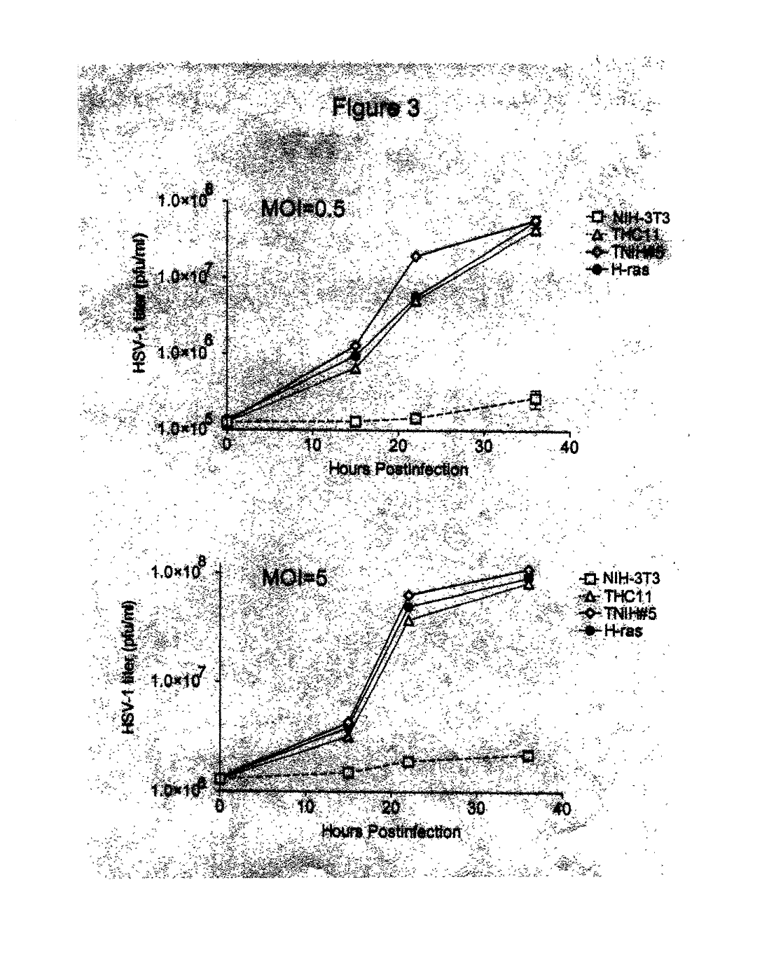

FIG. 3: A comparison, by plaque titration, of HSV-1 virus yield from HSV-1 infected NIH-3T3 cells and NIH-3T3 cells that are transformed with various oncogenes. The upper panel (A) shows the results using a MOI of 0.5 PFU/cell, whereas the lower panel (B) shows the results using a MOI of 5.0 PFU/cell.

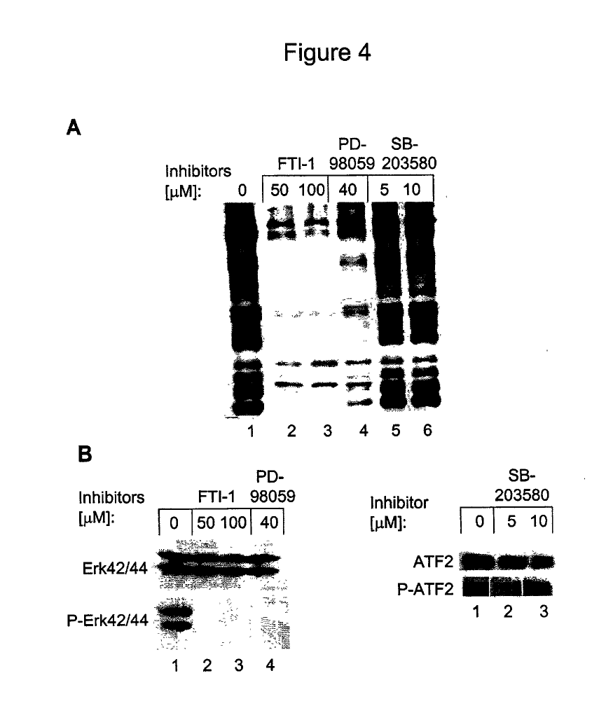

FIG. 4: Panel A shows a comparison, by immunoblotting of viral proteins, of the effect of FTI-1, an ERK pathway inhibitor (PD98059) and a p38 pathway inhibitor (SB203580) on the ability of HSV-1 to infect H-ras transformed cell lines. Panel B shows the effect of FTI-1 and PD98059 on Erk42/44 phosphorylation, and the effect of SB203580 on ATF2 phosphorylation.

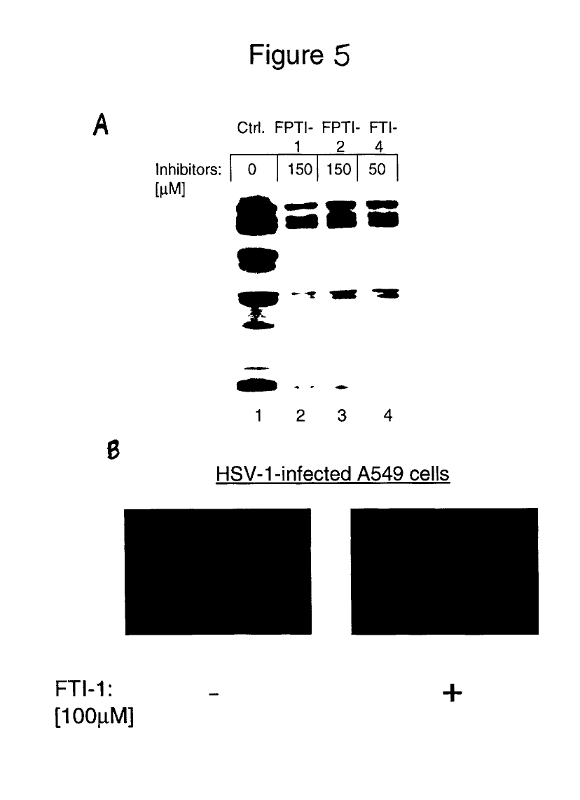

FIG. 5: Panel A shows the effect of three different inhibitors, FPTI-1, FPTI-2 and FTI-4 on HSV-1 viral protein synthesis, as compared to control (H-ras transformed) cells that have not been exposed to inhibitors. Panel B shows the effect of 100 μM FTI-1 on HSV-1-infected A549 cells, using immunofluorescence.

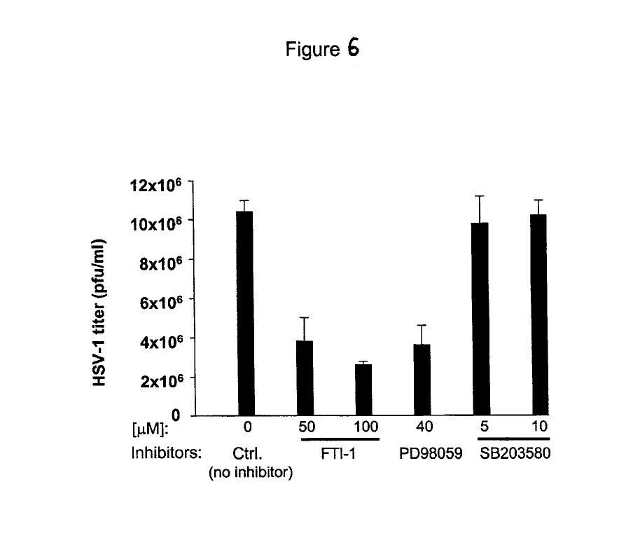

FIG. 6: HSV-1 virus yield from H-ras transformed cell lines in the presence of FTI-I in two different concentrations, PD98059 and SB203580 in two different concentrations.

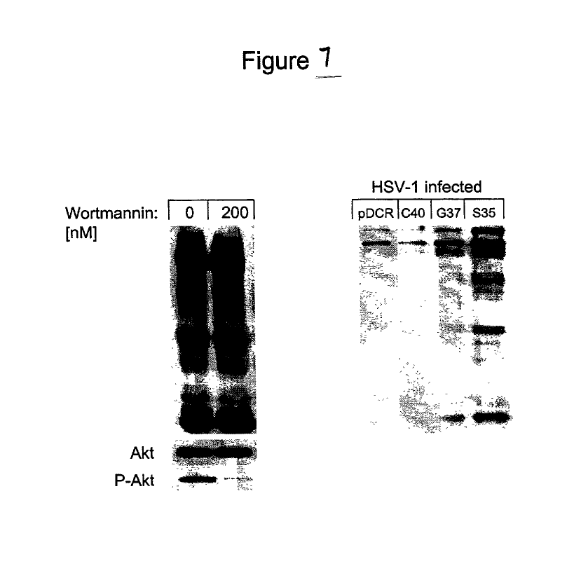

FIG. 7: The left panel shows a comparison, by immunoblotting of viral proteins, of the effect of Wortmannin, an inhibitor of P13-kinase on the ability of HSV-1 to infect H-ras transformed cell lines, and the effect of Wortmannin on Akt phosphorylation. The right panel shows the ability of HSV-1 to infect NIH-3T3 cells that express Ras effector domain mutants. The mutant cell lines are V12C40(C40), V12G37(G37) and V12S35(S35).

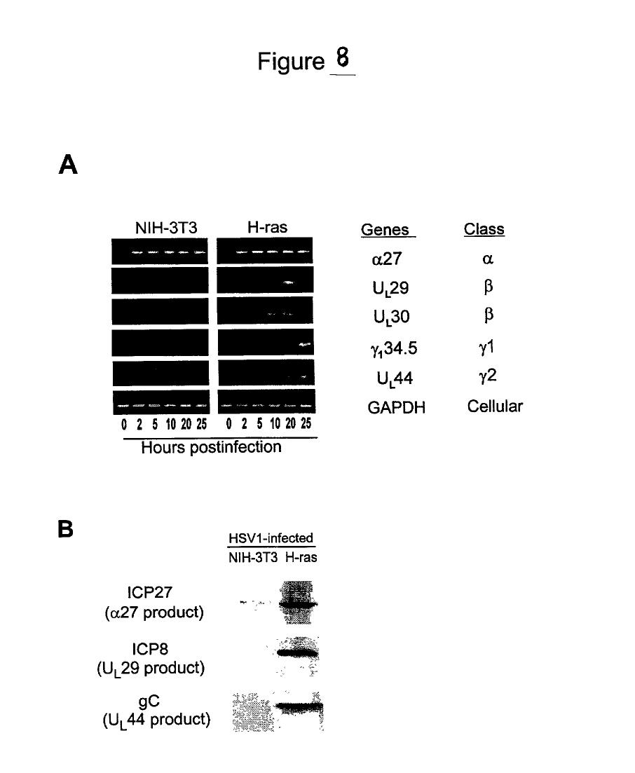

FIG. 8: Panel A shows quantitative RT-PCR of early, middle and late HSV-1 gene expression in HSV-1 infected NIH 3T3 cells and H-ras transformed cells. Panel B shows a comparison, by immunoblotting, of the level of ICP27, ICP8 and gC, in HSV-1 infected NIH-3T3 cells and H-rastransformed NIH-3T3 cells.

FIG. 9: Panel A shows an immunoblot comparison showing the early detection of HSV-1 infection in THC-11 and H-rastransformed cell lines as compared to A549 cell line. Panel B shows that infection of H-ras cells by HSV-1 can be detected using immunofluorescence with anti-gC antibody as early as 8 hours after the cells have been exposed to the virus.

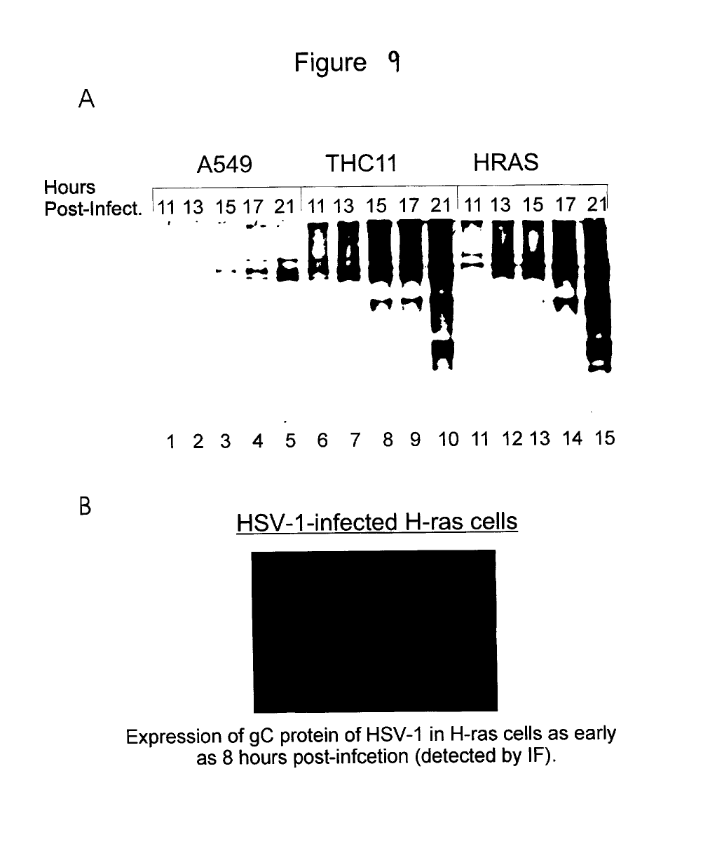

FIG. 10: Panel A shows a comparison, by immunoblotting, of the phosphorylation state of PKR and eIF-2 α in HSV-1 infected and uninfected cell lines (NIH-3T3 and oncogene-transformed NIH-3T3 cell lines). Panel B shows a comparison, by immunoblotting, of the phosphorylation state of PKR in HSV-1 infected and uninfected cell lines (NIH-3T3 and oncogene-transformed NIH-3T3 cell lines) after exposure to FTI-1 and PD98059 (left panel). A comparison, by immunoblotting, of phosphorylation state of PKR in HSV-1 infected NIH-3T3 cells and MEF cells (right panel).

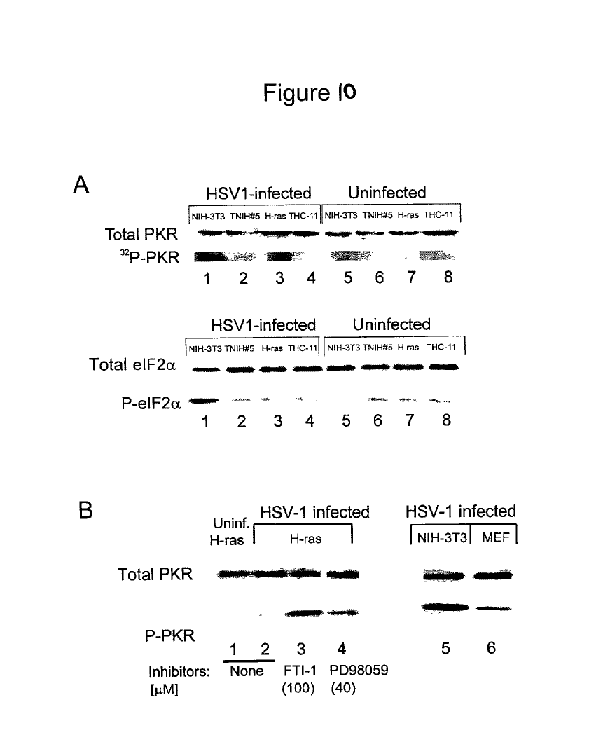

FIG. 11: A comparison, by immunoblotting, of the level of viral protein synthesis in NIH-3T3 cells and oncogene-transformed control cell lines, after infection with R3616.

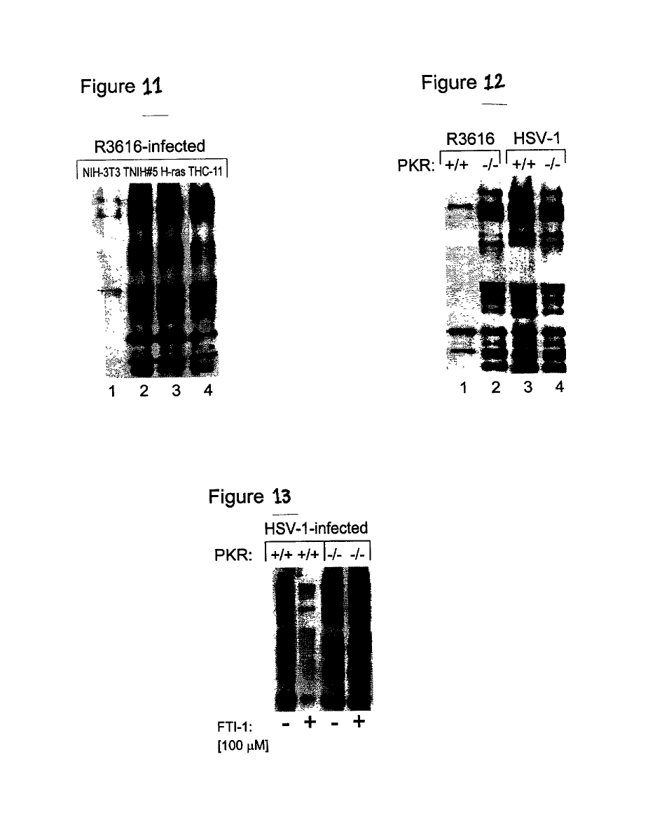

FIG. 12: A comparison, by immunoblotting, of the level of viral protein synthesis after infection of PKR^{+/+} and PKR^{-/-} fibroblasts with HSV-1 and R3616.

FIG. 13: A comparison, by immunoblotting, of the level of viral protein synthesis after infection of PKR and PKR fibroblasts with HSV-1 and exposure to FTI-1.

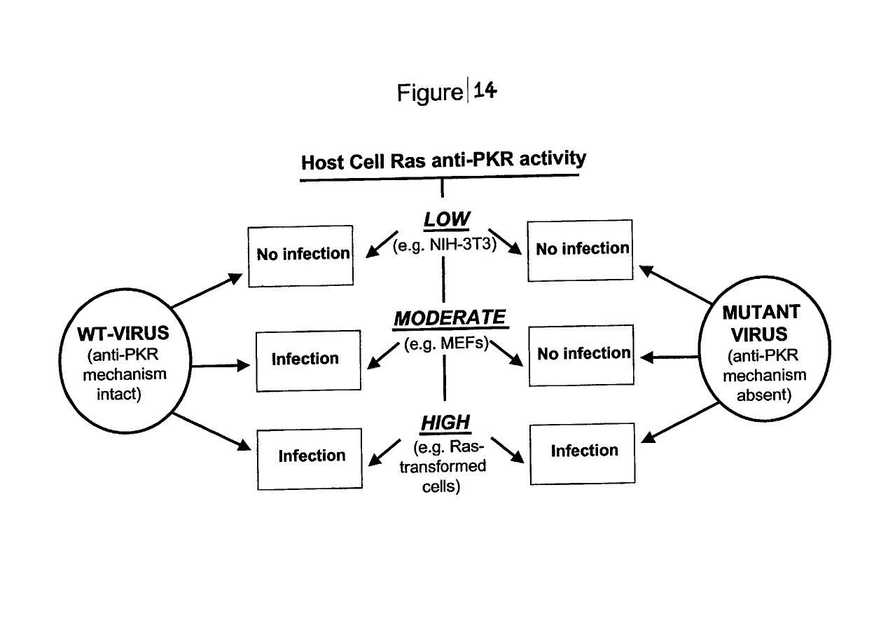

FIG. 14: Model demonstrating how the host cell and viral anti-PKR mechanisms influence of normal and transformed cells by wild-type and mutant HSV-1 virus.

### DETAILED DESCRIPTION

The invention pertains to methods of, or pharmaceutical compositions for, treating or preventing viral infections, preferably human herpes virus infections, and more preferably primary or recurrent HSV infections. The invention also pertains to methods of, or diagnostic kits for, diagnosing viral infections by detecting virus in a specimen.

Unless otherwise indicated, all terms used herein have the same meaning as is commonly understood by one skilled in the art of the present invention. Practitioners are particularly directed to Current Protocols in Molecular Biology (Ausubel) or Maniatis etal., Cold Spring Harbor, N.Y., Cold Spring Harbor Laboratory (1990), the contents of which are incorporated herein by reference, for terms of the art.

As used herein the following terms have the following meanings: “active infection” means a state where the virus is replicating within a cell, causing cytopathic effects, viral protein synthesis or virus output. In the appropriate context, active infection means a physiological state in a patient where the virus is being produced or actively shedding or where there are physical manifestations of viral infection such as lesions or sores.

“anti-PKR activity” includes any activity which has the consequence of opposing, countering or acting contrary to, the PKR system, or the effect of the PKR system. The anti-PKR activity may originate from a cellular or viral element and can be directed against PKR itself, or elements upstream or downstream of PKR, such as, for example eIF-2 α. “anti-viral effective dosage ” describes a dosage of an agent which inhibits the growth, replication and/or elaboration of the herpes virus in the patient or, in the case of prophylactic methods, substantially prevents the infection in the host (patient) in the first instance.

“excipient” includes a substance that is associated in any physical form with an anti-herpes agent, such that the anti-herpes agent is adsorbed and absorbed to, adhered to, dispersed, suspended or dissolved within such an excipient. The excipient may be solid, semi-solid, or liquid.

“farnesyl transferase inhibitor” includes any molecule, compound or composition, synthetic or natural, which has the effect of decreasing the activity of farnesyl transferase, either directly or indirectly. As used herein, “farnesyl transferase inhibitor ” includes Type 1-4 farnesyl transferase inhibitors and prodrugs of these inhibitors such as Type 1-3 farnesyl protein transferase inhibitors “herpes or herpes virus” includes herpes simplex virus type 1 (HSV-1); herpes simplex virus type 2 (HSV-2); varicella-zoster virus (VZV); cytomegalovirus (CMV); Epstein-Barr virus (EBV) and various other human herpes viruses (HHV) such as HHV-6, HHV-7 and HHV-8.

“HSV” means herpes simplex virus, and includes both type-1 and type-2.

“HSV-1 infected” refers to cells that are exposed to HSV-1 virus, and does not incorporate any reference as to whether the cells are permissive or non-permissive to the virus. In the appropriate context, HSV-1 infected may mean cells that are exhibiting signs of active infection. “infection ” includes active infection of permissive cells by a virus and latent infection with the virus.

“oncogene-transformed cell lines” means cell lines that are transformed with an oncogene.

“patient” refers to an animal, generally a human who can be infected with a herpes virus and experience the manifestations of a herpes viral infection. The patient may be a human or other animal, depending upon the type of virus.

“Ras pathway ” includes signal transduction pathways that are downstream of receptor tyrosine kinases (RTK”s) such as epidermal growth factor receptors or non-receptor kinases (nRTK”s) such as the srcfamily kinases, which can lead to the activation of Ras and its downstream elements. As used herein, “Ras pathway” includes other biochemical pathways which lead or can lead to the activation of Ras, or its upstream or downstream elements (e.g. through activation of any G proteins, P13 kinase, PKC, Calcium, FAK etc.). Pathways downstream of Ras which are included in this definition include the MAPK cascade consisting of Raf isoforms, MEK1/2, and ERK1/2.

“non-permissive cells” refers to cells that do not support virus growth as demonstrated by the substantial lack of cytopathic effects, viral protein synthesis or virus output after exposure to a virus.

“permissive cells” refers to cells that support virus growth as demonstrated by the induction of substantial cytopathic effects, substantial viral protein synthesis or virus output after exposure to a virus.

As described herein, the applicants have discovered that HSV-1 exploits an activated tyrosine receptor kinase/Ras pathway for infection. NIH-3T3 cells, which are non-permissive to HSV-1 infection, become permissive when transformed by the oncogenes v-erb B, Sos, or H-Ras. These oncogenes are all activators of the Ras signaling pathway. Permissiveness of these cells to HSV-1 infection is defined by the induction of cytopathic effects, enhanced viral protein synthesis, and/or production of progeny HSV-1 virus. The applicants have demonstrated that cells non-permissive to HSV-1 infection inhibit viral replication at the protein translational level by phosphorylated PKR. Cells permissive to HSV-1 infection have an activated Ras pathway that dephosphorylates or prevents the phosphorylation of PKR, which allows viral protein translation to proceed.

The applicants have shown that the farnesyl transferase inhibitors FTI-1 and FPTI-II effectively block HSV-1 infection in H-ras transformed NIH-3T3 cells, which otherwise is permissive to HSV-1 infection. Posttranslational farnesylation of Ras is necessary for association of Ras with the plasma membrane, and is known to be important for the initiation of downstream events, including three distinct MAPK cascades, (eg. ERK, JNK) [3-7]. The enzyme farnesyl transferase covalently links a farnesyl group (15 carbon isoprenoid) to a cysteine residue located in the carboxy terminal CAAX motif of Ras, allowing the latter to be anchored to the plasma membrane. Farnesyl transferase inhibitors have been developed as potential anticancer agents that block farnesylation and thus inhibit the function of oncogenic Ras. Without being limited to a theory, it appears that the inhibition of HSV-1 replication by farnesyl transferase inhibitors means that HSV-1 infection requires an activated Ras pathway of the host cell.

Extracellular signals received by cell surface receptors are transformed into intracellular instructions that coordinate the appropriate cellular responses [6]. Nearly all cells use one or more MAPK (mitogen-activated protein kinase) cascades to accomplish this. The ERK pathway (extracellular signal regulated kinase is one such cascade, which acts downstream of Ras to regulate cellular growth [6, 8, 9]. Ras regulates the activity of Raf, a serine-threonine kinase in this pathway. Raf activates MEK1/2 [MAPK/ERK kinase], which activates ERK1/2, one of the latter members in a pathway that plays a role in cellular proliferation and differentiation.

The applicants have further shown that 1/2 activity is required for HSV-1 infection. PD98059 [10], an inhibitor of MEK1/2, partially blocked HSV-1 infection in H-ras transformed NIH-3T3 cells. Combined, these results show that the ERK pathway is involved in HSV-1 infection. The applicants have further shown that neither SB203580 [11] a specific inhibitor of p38 kinase, or Wortmannin [12] a specific inhibitor of P13-kinase, had any measurable effect on HSV-1 infectivity.

The applicants have studied the effect of three ras effector mutant cell lines V12C40, V12G37 and V12S35 [13-15] on the ability of ras to increase the infectivity of NIH-3T3 cells to HSV-1. All three cell lines have a common activating G12V mutation as well as one other unique mutation in Ras, causing them to activate distinct pathways downstream of Ras. Mutant V12G37 is unable to signal via the RAF/ERK and the P13-kinase pathway, but allows signaling via the RAL-GDS pathway. The V12C40 mutation disrupts signaling via the RAF/ERK and the RAL-GDS pathways, but does not affect the P13-kinase pathway. The V12S35 mutant cannot signal through the RAL-GDS and the P13-kinase pathway, but can do so via the RAF/ERK pathway. Our results show that the V12S35 mutant is considerably more permissive to HSV-1 infection than the other two mutants, suggesting a more significant role of the RAF/ERK pathway (as compared with the P13-kinase or the RAL-GDS pathway) in the infection process.

The applicants have demonstrated that non-permissiveness to HSV-1 infection in cells is predominantly at the level of α gene translation. The viral immediate early transcript α 27 accumulates to comparable levels in both non-permissive and permissive cell lines. However, the viral β- and γ-class genes, whose transcription is dependent upon the presence of α gene products, are much less abundant in non-permissive cells. The α 27 gene product, ICP27, is also much less abundant in non-permissive cells than in permissive cells. Without being limited to a theory, it appears that the α 27 transcripts are not translated in non-permissive cells, therefore downstream events, such as β and γ gene expression, do not occur, resulting in abortive HSV-1 infection.

The mechanism of host cell non-permissiveness to viral infection is correlated with viral-transcript induced phosphorylation of an approximately 65 kDa cellular protein, determined to be a double-stranded RNA-activated protein kinase (PKR) [16]. Phosphorylated PKR will, in turn, phosphorylate eIF-2 α —a translation initiation factor that efficiently inhibits viral gene translation. Permissiveness to viral infection then, is correlated to lack of phosphorylation of PKR, which means that eIF-2 α is not phosphorylated, and therefore translation of viral genes can proceed.

The applicants have discovered that permissiveness to HSV-1 infection is correlated to the lack of PKR phosphorylation in transformed cells. These cells, which are permissive to HSV-1 have lower levels of phosphorylated PKR than do non-permissive cells. It appears that HSV-1 permissive cells either lack the ability to phosphorylate PKR, or have an enhanced ability to dephosphorylate PKR, or its downstream elements, for example eIF-2 α.

Attenuated mutants of HSV-1 are being tested as anti-cancer agents in clinical trials, but the exact mechanism of their anti-cancer effect is unknown. HSV-1 mutant R3616 [17] contains deletions of both copies of the viral γ₁ 34.5 gene. The gene product of γ₁ 34.5 (called ICP34.5) presumably forms a complex with protein phosphatase 1, and redirects its activity to dephosphorylate eIF-2 α [18-20]. Since PKR phosphorylates eIF-2 α, ICP34.5 therefore plays an antagonistic role to PKR—i.e. it acts “anti-PKR”, and mutant R3616 is a virus that has lost its inherent anti-PKR mechanism.

The applicants have discovered that the reason these mutant HSV-1 viruses selectively kill cancer cells is that elements of the Ras pathway inactivate PKR or inhibit the phosphorylation of PKR. If inactivated, PKR is unable to phosphorylate eIF-2 α, and viral infection proceeds to kill the cancer cells. In normal cells, the Ras pathway is not up-regulated and therefore anti-PKR activity is not manifested.

As a result PKR is active and phosphorylates eIF-2 α, which blocks translation of viral transcripts thereby inhibiting viral infection.

Therefore, HSV-1 exploits both the host cell and/or viral anti-PKR mechanism for infection (see FIG. 14). When the viral intrinsic anti-PKR mechanism is weakened or destroyed, as is the case with R3616, a stronger host cell anti-PKR “arm” is required to compensate for this loss, such that productive infection can result. Host cells with an activated Ras pathway (e.g. certain cancer cells) would therefore support viral growth, whereas normal cells, whose Ras pathway activity does not normally reach the threshold level to inactivate PKR, will not be able to support viral growth. Thus, viruses whose intrinsic anti-PKR mechanism is rendered ineffective would be “attenuated”, in that they are incapable of infecting normal cells, but become cytolytic in cells with a strong anti-PKR activity, as would be found in cancer cells.

Based upon these discoveries the applicants have developed methods and compositions for treating or preventing virus infections wherein the virus utilizes the Ras pathway or otherwise exploits a host cell anti-PKR mechanism for infection and for diagnosing such virus infections.

Treatment of Herpes Virus Infectionsin one embodiment, this invention relates to a method of treating or preventing herpes virus infections using pharmaceutical compositions that contain an anti-viral effective dosage of an anti-herpes agent which is administered to a patient suffering from a viral infection, or at risk for contracting a viral infection, in order to treat or prevent the viral infection in the patient.

In one aspect of this invention, primary and recurrent herpes virus-caused lesions and sores of the skin and mucosa are treated with a topical therapy comprising administration of an inhibitory effective amount of the anti-herpes agent to a human suffering from a herpes virus infection. The area to be treated may include, the lips, eyes, mouth, genital and anal area, and other areas accessible to topical administration which may be the site of a herpes lesion or sore.

In another aspect of this invention, primary and recurrent herpes virus-caused infections that may or may not be associated with lesions, are treated with a pharmaceutical composition that is used for regional or systemic administration of an inhibitory effective amount of the anti-herpes agent. These types of infections would include neonatal disseminated infections, encephalitis, respiratory distress syndrome or acute-onset bronchspasm. As used herein, “regional” administration includes administration to areas such as the peritoneal space, pelvic cul-de-sac, reproductive tract, central nervous system, perineum, respiratory tract and rectovaginal region. “Systemic” administration refers to the circulatory system and regions outside the spaces described above.

Pharmaceutical compositions of this invention contain an “anti-herpes agent”. An anti herpes agent is an agent which is any molecule, compound or composition which inhibits, reduces or eliminates replication of the herpes virus in cells, reduces or eliminates the manifestation of herpes virus infections in patients, or which leads to a decrease in the host cell permissiveness to HSV infection. An antiherpes agent according to the present invention acts by inhibiting Ras or a component of the Ras pathway, including Raf, MEK1/2 or ERK1/2; or by promoting or allowing PKR phosphorylation, activity or function; or by promoting or allowing PKR-mediated downstream events, such as eIF-2 α phosphorylation, activity or function (eg. PD98059; FTI”s, U0124, U0125 or U0126). An anti-herpes agent may also be effective against other viral infections, if the virus exploits an activated Ras pathway or otherwise exploits a host cell anti-PKR activity for infection. Examples of such other viruses include reovirus, vesicular stomatitis viruses (VSV) and influenza viruses.

In one embodiment, the anti-herpes agent may be a farnesyl transferase inhibitor. Those skilled in the art are aware that there are many different inhibitors of farnesyl transferase, and that pro-drugs of these inhibitors or farnesyl protein transferase inhibitors may also have the same effect as a farnesyl transferase inhibitor. Examples of known farnesyl transferase inhibitors include FTase Inhibitor I, FTase Inhibitor II, FTase Inhibitor III, FTase Inhibitor IV, FTI 276, FTI 277, FPT Inhibitor I, FPT Inhibitor II and FPT Inhibitor III and FTS. Suitable farnesyl transferase inhibitors may be described in U.S. Pat. No. 6,156,746. As well, there are other ways of modifying the activity of farnesyl transferase, including but not limited to, post-translational modification, neutralizing antibodies or anti-sense RNA. Farnesyl transferase interacts with other proteins and molecules within the cell, that act both upstream and downstream of it. The scope of this invention includes any pharmaceutical composition that modifies the activity or effect of farnesyl transferase, either directly or indirectly, if this composition has the effect of decreasing the permissiveness of cells to herpes virus infection. The specific embodiments of this invention as described herein are not intended to limit the applicability of the principles involved.

In another embodiment, the anti-herpes agent may be an inhibitor of the ERK pathway such as PD98059, U0124, U0125 or U0126. Those skilled in the art are also aware that Ras activity may be modified by means other than modification of the activity of farnesyl transferase, and that Ras interacts with other proteins and molecules within the cell, that act both upstream and downstream of Ras. For example, other agents that inhibit membrane association of Ras, agents that block or decrease the activity of receptor tyrosine kinases, adaptor molecules, GEF”s (guanine exchange factors) or agents that enhance the activity of GAP”s (GTPase activating proteins), or agents that affect the appropriate localization or activation of Raf can all affect the activity or the effect of the activity of Ras or the Ras pathway. The scope of this invention includes any pharmaceutical composition that modifies the activity or effect of Ras or the Ras pathway, either directly or indirectly, if this composition has the effect of decreasing the permissiveness of cells to herpes virus infection. The specific embodiments of this invention as described herein are not intended to limit the applicability of the principles involved.

Those skilled in the art are aware that there are, or may be, other means of modifying the activity of any member of the MAPK pathway, including the ERK pathway. These other means include post-translational modification, neutralizing antibodies or the use of an anti-sense RNA. As well, members of the MAPK pathway interact with other proteins and molecules within the cell, that act both upstream and downstream of the pathway. The scope of this invention includes any pharmaceutical composition that modifies the activity or effect of the MAPK pathway, either directly or indirectly, if that composition has the effect of decreasing the permissiveness of cells to herpes virus infection. The specific embodiments of this invention, as described herein are not intended to limit the applicability of the principles involved.

The present invention provides pharmaceutical compositions which may contain between 0.01% and 30%, (weight percentage) of an anti-herpes agent as described above and one or more pharmaceutically acceptable excipients. In making the compositions of this invention, the anti-herpes agent is usually mixed with and/or diluted by, one or more excipients. Alternatively, or in addition, the anti-herpes agent may be enclosed within a carrier, in the form of a capsule, sachet, paper or other container. As the excipient may be solid, semi-solid or liquid, the resultant composition may also be solid, semi-solid or liquid.

Some examples of suitable excipients include lactose, dextrose, sucrose, sorbitol, mannitol, starches, gum acacia, calcium phosphate, alginates, tragacanth, gelatin, calcium silicate, mircrocrystalline cellulose, polyvinylpyrrolidone, cellulose, sterile water, syrup and methyl cellulose. The pharmaceutical compositions can additionally include: lubricating agents such as talc, magnesium stearate and mineral oil; wetting agents; emulsifying and suspending agents; preserving agents such as methyl- and proplyhydroxy-benzoates; sweetening agents and flavoring agents. The invention can be formulated so as to provide quick, sustained or delayed release of the anti-herpes agent after administration to the patient by employing procedures known in the art. Other components may be added to the pharmaceutical composition based upon the related drug delivery system, to improve the pharmacokinetics or pharmacodynamics of the composition.

The pharmaceutical compositions of the present invention can include other active ingredients. For instance, drugs that are commonly used to treat herpes, such as nucleoside analogs, or pain relieving drugs such as acetaminophen may be added to the composition. Additionally, agents or chemical additives that enhance the anti-viral activity of the anti-herpes agent, or more than one anti-herpes agent, can be included in the pharmaceutical composition.

Topical pharmaceutical compositions in the form of an ointment, cream, gel, solution, lotion, emulsion, aerosol, powder, or other topical vehicle, including a sponge, suppository or stick, are designed and prepared such that a therapeutically effective amount of the anti-herpes agent is brought into contact with diseased tissue. An excipient or carrier may take a wide variety of forms depending on the form of preparation desired for topical administration, which includes intrarectal or intravaginal administration. In preparing pharmaceutical compositions in topical dosage form, any of the usual pharmaceutical media may be used.

To create a viscous ointment, for example, de-ionized water, oil and an emulsifier are intermingled to create an emulsion. A suitable oil for such a purpose is petrolatum and a suitable emulsifier is wax NF. It is also desirable to include in the ointment a preservative, such as methyl paraben or propyl paraben as well as a humectant, such as propylene glycol.

For preparing solid compositions such as tablets, pills and capsules, the anti-herpes agent is mixed with excipient such that the anti-herpes agent is present homogeneously throughout the composition, and therefore the composition may be subdivided into equally effective unit dosage forms. Alternatively, the antiherpes agent may be localized to only a portion of a tablet if it is desired to combine the activity of the anti-herpes agent with one or more other active ingredients.

The tablets or pills may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, flavored syrups, aqueous or oil suspensions and flavored emulsions with edible oils, such as corn oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described herein. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension or powder compositions maybe administered, preferably orally or nasally, from devices which deliver the formulation in an appropriate manner.

Other suitable formulations for use in the present invention can be found in Remingtons Pharmaceutical Sciences the contents of which are incorporated herein. While the invention has been described in connection with certain preferred embodiments, these are not meant to limit the invention, which covers all alternatives, modifications and equivalents as may be included within the scope of this invention. The preferred embodiments are presented to provide what is believed to be the most useful and readily understood description of the invention, as well as of the principles and conceptual aspects of the invention.

Treatment with the pharmaceutical compositions of this invention begins either once a patient is known to have been exposed to the herpes virus, when the symptoms of infection are apparent in the patient, either visibly or by other indications such as pain, when the patient is suspected to have an active herpes infection, or when a patient is diagnosed with an active herpes infection. Treatment continues until such time as the risk of infection is over or until the symptoms of the virally-caused infection have disappeared.

Diagnosis of Herpes Virus Infectionln accordance with the present invention, a method and diagnostic kit are provided for detecting infectious herpes virus in a specimen, using a genetically engineered cell line for such method. The method employs a cell line that has been transformed with a gene, including an oncogene, that activates the Ras pathway.

In a preferred embodiment, cell lines are modified by transfection with DNA constructs that contain oncogenes that are activators of the Ras pathway. A preferred cell line is NIH-3T3, but other cell lines can be used, including NR6 cells or Swiss 3T3 cells. The cell lines can be from any mammalian organism. The DNA construct to be transfected, which is preferably a plasmid, contains the entire coding region of the oncogene, or sufficient sequence thereof such that the oncogene will be active within the cell. In this embodiment, preferred oncogenes are v-erbB, Sos and H-ras. However, other oncogenes, and genes other than oncogenes can be used, such as, for example, non receptor tyrosine kinases, and receptor tyrosine kinases and their downstream elements, including raf, erk, or any other MEK or MEKK or MAP kinase. The oncogene sequence is inserted downstream of a gene promoter which will drive expression of the oncogene in the cell line that is to be transfected. Preferred is a continuous promoter such as CMV; HSV-TK or SV-40 but other promoters, such as inducible promoters, can be used.

The DNA construct is transfected into the appropriate cell line such that stable integration of the gene into the chromosome of the cell occurs, using standard and routine methods known to those skilled in the art.

In order to detect the presence of an infectious herpes virus in a specimen, a cell line that has been stably transfected with an oncogene, as described above, is inoculated with a specimen by placing an aliquot of each in a suitable standard culture medium in standard culture vessels. The specimen may be any material which can be placed into a fluid or fluid from the patient, such as blood, semen, nasopharyngeal swabs, cerebrospinal fluids and the like. The cell line and the specimen are cultured for a sufficient period of time to allow the herpes virus infectious cycle to proceed. The time frame within which active infection can be detected in the cell lines that are used in the method of this invention is as little as 8 hours. Therefore, compared to the cells lines that are currently used in the art to detect infection, in which a minimum of 20 hours is needed before infection can be detected, the cells lines used in the method of this invention have dramatically reduced the time frame for diagnosis.

Viral infection can also be detected by immunofluorescence using for example the Microtrack HSV Culture Identification Kit, which will also allow type-specific identification of the HSV virus in the sample. These methods are currently used by those skilled in the art, however because the cell lines used in the method of this infection are more sensitive to infection, the time frame for immunological detection is greatly reduced. The Applicants have used immunoassays with HSV-1 gC antibodies to detect viral infection as early as 8-10 hours post inoculation.

The α proteins of HSV-1 (eg. ICP27, ICP4, ICP0) or the β proteins (eg. ICP8) are produced as early as 4 hours post-infection. Since the cells used in the method of this invention produce the complete spectrum of HSV-1 proteins much earlier than cells lines currently used in the art, another embodiment of this invention uses an antibody that detects only an early viral protein, such as the mouse anti-ICP27 antibody used in the Examples herein, to detect HSV infection in a sample. This particular embodiment may be useful, for example when very rapid diagnosis is needed, as in the instance of life-threatening HSV infections.

As will be apparent to those skilled in the art, various modifications, adaptations and variations of the preceding and foregoing specific disclosure can be made without departing from the scope of the invention claimed herein. The following examples are intended only to illustrate and describe the invention rather than limit the claims which follow.

EXAMPLES In the examples below, the following abbreviations have the following meanings. If an abbreviation is not defined, it has its generally accepted meaning: α-MEM=α-modified Eagle”s medium β-ME=β-mercaptoethanolDMEM=Dulbecco”s modified Eagle”s mediumDMSO=dimethylsulphoxideDTT=dithiothrietolERK kinase=mitogen-activated extracellular signal-regulated kinaseFBS=fetal bovine serumFPTI-II=farnesyl protein transferase inhibitor IIFTI=farnesyl transferase inhibitorFTS=farnesyl thiosalicylic acidGAPDH=glyceraldehyde-3-phosphate dehydrogenaseHEPES=NSulfonic AcidHRP=horseradish peroxidaseMAPK=mitogen activated protein kinaseMOI=multiplicity of infectionPAGE=polyacrylamide gel electrophoresisPBS=phosphate buffered salinePFU=plaque forming unitsphosph-MAPK =phosphorylated-MAPKPKR=double-stranded-RNA-activated phosphokinaseRT-PCR=reverse transcript-polymerase chain reactionSDS=sodium dodecyl sulphateGeneral MethodsCells and virusesParental NIH-3T3 and NIH-3T3 cells transfected with the Harvey-ras (H-ras) oncogene were a generous gift of Dr. Douglas Faller (Boston University School of Medicine). NIH-3T3 cells along with their Sos-transformed counterparts (designated TNIH#5) were a generous gift of Dr. Michael Karin (University of California, San Diego). Dr. H. J. Kung (Case Western Reserve University) kindly donated parental NIH-3T3 cells along with NIH-3T3 cells transfected with the v-erbBoncogene (designated THC-11). All cell lines were grown in DMEM containing 10% FBS.

The PKR⁺/⁺ and PKR⁻/⁻ mouse embryo fibroblasts were obtained from Dr. B. R. G. Williams (the Cleveland Clinic Foundation) and were grown in α-MEM containing 10% FBS and antibiotics, as described [21].

Wild-type HSV-1 strain F [HSV-1(F)] and mutant HSV R3616 were both gifts from B. Roizman and have been described [17, 22-24].

Immunoflourescent analysis of HSV-1 infectionNIH-3T3, TNIH#5, THC-11, and H-ras cells were grown in 8-well slide chambers (Falcon) and infected with HSV-1 at a MOI of 0.5 PFU/cell, or mock-infected by application of PBS to the cells in an identical fashion as the administration of virus to the cells. At 20 hours after infection, the cells were fixed in 100% acetone for 10 min and then left at room temperature to dry. The fixed and dried cells were then incubated with a fluorescein-labeled mouse monoclonal antibody to HSV-1 gC antigen (SyvaMicrotak from Behring) for 30 min at 37° C. The slides were then washed with distilled water, dried, mounted in 90% glycerol containing 0.1% phenylenediamine, and viewed with a Zeiss Axiophot microscope on which a Carl Zeiss camera was mounted. The magnification for all pictures was 200X.

Western blot (Immunoblot) analysisinfected cells were lysed with the single detergent lysis buffer [50 mM Tris (pH 8.0), 150 mM NaCI, 0.02% sodium azide, 100 μg/ml phenylmethy-sulfonyl fluoride, 1 μg/ml aprotinin, and 1% Triton X-100], normalized for the amount of total protein and subjected to SDS-PAGE, followed by electroblotting onto nitrocellulose paper. The membrane was then washed and incubated with the primary antibody [rabbit antibody against all HSV-1 antigens (Dako, CA.; 1:20,000); mouse anti-ICP27 or anti-gC antibody (Rumbaugh-Goodwin Institute, FL; 1:1,000); rabbit anti-ICP8 antibody (from Dr. Paul Olivo, Washington University, St. Louis; 1:1,000); mouse anti-PKR and mouse anti-eIF-2 α antibody (Santa Cruz; 1:1,000); rabbit anti-phospho-PKR and rabbit antiphospho-eIF-2 α antibody (Biosource, CA; 1:1,000) followed by HRP-conjugated secondary antibody (1:2,000). After extensive washing, the blot was exposed to Lumigel detection solution (New England Biolabs) and subjected to autoradiography.

Uninfected H-ras cells were used to demonstrate the effects of FTI-1, PD98059 on ERK1/2 phosphorylation, SB203580 on ATF-2 phosphorylation, and Wortmannin on Akt phosphorylation. Briefly, subconfluent monolayer cultures were lysed with the recommended SDS-containing sample buffer, the lysate was subjected to SDS-PAGE and electroblotted onto nitrocellulose paper. Blots were probed with anti-ERK1/2 or anti-phospho-ERK1/2 antibodies (for samples treated with FTI-1 or PD98059), anti-ATF-2 or anti-phospho-ATF-2 (for samples treated with SB203580), and anti-Akt and anti-phospho-Akt (for samples treated with Wortmannin). The antibody kits were purchased from New England Biolabs (MA). FTI-1, PD98059, SB203580 and Wortmannin were purchased from Calbiochem (CA).

Polymerase chain reaction Cytoplasmic RNA from infected cells was isolated using the RNeasy kit by Quiagen (CA). Briefly, at various times post-infection, monolayers of cells (approximately 1×10⁷ cells) were lysed using the RLN buffer (50 mM Tris-Cl pH 8.0, 140 mM NaCI, 1.5 mM MgCl₂0.5% NP-40, 1 mM DTT and 1000 U/ml RNasin). After removal of the nuclei by centrifugation, the supernatant was mixed with buffer RLT and ethanol, and applied to an RNeasy spin column. The column was subsequently washed and RNA was eluted in water. Equal amounts of cytoplasmic RNA from each sample were then subjected to RT-PCR using random hexanucleotide primers (Pharmacia) and reverse transcriptase (GIBCO-BRL) according to the manufacturers” protocol. The cDNAs from the RT-PCR step was then subjected to selective amplification of cDNAs of α 27, U^{L}29, U_{L}30, γ₁34.5, and U_{L}44. For α 27 the primers 5”-CTGGAATCGGACAGCAGCCGG-3” and 5”-GAGGCGCGACCACACACTGT-3” were used, which produced the predicted 222 bp fragment. For U^{L}2 the primers used were 5”-GCGCCCCATGGTCGTGTT-3” and 5”-CTCCGCCGCCGAGGTTC-3”, which produced the predicted 206 bp fragment. For U_{L}30, the primers 5”-ATCAACTTCGACTGGCCCTTC-3” and 5”-CCGTACATGTCGATGTTCACC-3” were used, which produced the predicted 180 bp fragment. For γ₁34.5, the primers used were 5”-CTCGGAGGGCGGGACTGG-3” and 5”-GCGGGAGGCGGGGMTAC-3”, which produced a predicted 282 bp fragment. For U_{L}44, the primers used were 5”-GCCGCCGCCTACTACCC-3” and 5”-GCTGCCGCGACTGTGATG-3”, which produced a predicted 661 bp fragment. As a PCR and gel loading control, GAPDH primer 5”-CGGAGTCAAGGATTTGGTCGAT-3” and 5”AGCCTTCTCCATGGTGGTGAAGAC-3” were used to amplify a predicted 306 bp GAPDH fragment. Selective amplification of the various cDNAs was performed using HotStarTaq DNA polymerase (Quiagen) in a MiniCycler PTC-150 (MJ-Research). PCR was carried out for 30 cycles, with each cycle consisting of a denaturing step for 1 min at 94° C., an annealing step for 2 min at 60° C., and a polymerization step for 2 min at 72° C. The PCR products were separated on a 1.5% agarose gel impregnated with ethidium bromide, and photographed under UV illumination with Polaroid 57 film.

### EXAMPLE 1

Activators of the Ras Pathway Augment HSV-1 Infection EfficiencyNIH-3T3 cells are known to be poorly infectible with HSV-1. It was of particular interest to determine whether NIH-3T3 cells that were transformed with oncogenes which are activators of the Ras pathway were equally as non-permissive to HSV infection.

Monolayers of NIH-3T3 cells, v-erbB-(THC-11), Sos-(TNIH#5), or H-ras transformed NIH-3T3 cells were exposed to HSV-1 (strain F) at a MOI of 0.5 PFU per cell. Cells were photographed 20 hours after infection in order to determine the cytopathic effects of the virus on these cells. Cells were then fixed, processed and reacted with a FITC-labeled mouse anti-HSV-1 gC antibody. As shown in FIG. 1, little or no morphological change could be detected in NIH-3T3 cells, which exhibited a typically flattened, spread out morphology with marked contact inhibition. In contrast, cells transformed with v-erbB, Sos or H-ras exhibited rounding or clumping, which are characteristic cytopathic effects of HSV-1 infection.

In order to determine whether viral proteins were being synthesized by the HSV-1 infected, oncogene-transformed cells, immunofluorescent microscopy of these cells was performed as described above. As shown in FIG. 1, the results show that virus proteins were detected only in a very small population of NIH-3T3 cells, whereas in oncogene-transformed cell lines, pronounced viral protein synthesis was observed. Scale bar, 150 μm.

The amount of HSV-1 protein synthesis in these cell lines was also determined by Western Blot analysis. Cells were infected as described above, or mock-infected, and were harvested at 10, 22 or 36 hours after infection (or mock-infection). Western blot analysis was performed on these samples using rabbit polyclonal antibody against all HSV-1 antigens, as described above, and the results are shown in FIG. 2. Lanes 13-16 show uninfected NIH-3T3, TNIH#5, H-ras, and THC-11 cells, respectively. As can be seen in FIG. 2, viral proteins were not present in NIH-3T3 cells, but were abundant in the oncogene-transformed NIH-3T3 cells. As all these cell lines have identical doubling times, the observed differences in the level of viral protein synthesis were not due to intrinsic differences in growth rates or translational efficiencies for these cell lines.

Similar results to the above were obtained by metabolic labeling with [³⁵S]-methionine. Briefly, at 12 hours post infection, the medium was replaced with methionine-free medium containing 0.1 mCi/ml [³⁵ S]-methionine. After further incubation for 36 hours, the cells were washed in PBS and lysed in PBS containing 1% Triton X-100, 0.5% sodium deoxycholate and 1 mM EDTA. The nuclei were removed by low speed centrifugation and the supernatants stored at 70° C. Aliquots (normalized for protein content) were electrophoresed through SDS-polyacrylamide gels and autoradiographed. The autoradiographs demonstrated that viral proteins were not present in NIH-3T3 cells but were abundant in the transformed NIH-3T3 cells.

The doubling times for uninfected NIH-3T3 and uninfected transformed cells are identical and they show very similar patterns and levels of cellular protein synthesis. Therefore, the observed differences in the level of viral protein synthesis could not be due to intrinsic differences in growth rates or translational efficiencies for these cell lines. This was further supported by the observation that NIH-3T3 cells could not be rendered more permissive even when infections were carried out at a lower cell density.

To determine whether the HSV-1 infected, oncogene-transformed NIH-3T3 cells were actually producing intact HSV-1 virus particles, HSV-1 virus yield from these cells was determined by plaque titration on Vero cells. Briefly, Vero cells were grown in 6-well multi-well plates. Different dilutions of virus, from 10⁻¹ to 10⁻⁶ are applied to each well and the cells are incubated for about 1.5 to 2 hours. Agar-DMEM (10%) was overlaid on the cells, which were then incubated for about 2 to 3 days, or until cytopathic effects are observed. The wells were then stained with neutral red. Areas containing plaques remained clear, whereas viable cells stained dark red. As shown in FIG. 3, THC-11, H-ras and TNIH#5 cells produced significant amounts of viral particles as early as 15 hours after infection, with titres between 1×10⁵ and 1×10⁶ PFU/ml. By 35 hours post-infection, the viral titres were between 1+10⁷ and 1×10⁸ pfu/ml. In contrast, the viral titres from infected NIH-3T3 cells at the 15- and 25-hr time points were below 1×10⁴ PFU/ml. FIG. 3 shows the HSV-1 virus yield from infected NIH-3T3, THC-11, TNIH#5 and H-ras cells using two different MOI”s (Upper panel: MOI=0.5 PFU”s/cell; lower panel: MOI=5 PFU”s /cell) This example illustrates that cell lines, once transformed with activators of the Ras pathway, become permissive to infection by HSV-1 and exhibit not only cytopathic effects from infection, but synthesize viral proteins and intact viral particles. In contrast, untransformed cell lines are non-permissive to HSV-1 infection.

### EXAMPLE 2

Ras-activated cells lines as a diagnostic tool for HSV-1 infection FIG. 3 shows that as early as 15 hours after infection with HSV-1, measurable virus titres are produced from cell lines that are transformed with activators of the Ras pathway.

FIG. 9, Panel “A” compares the sensitivity of detection of HSV-1 antibodies in three different cell lines. Cell line A549 (human lung carcinoma) is typically used in medical laboratories to diagnose HSV-1 infection. Cell line THC-11 is transformed with v-erbB, and H-ras cell lines are transformed with H-ras. All three cell lines were infected with HSV-1 at a MOI of 0.25-0.5. At 11, 13, 15, 17 or 21 hours post infection, cells were harvested and Western blot analysis was performed on these samples as described above. The results showed that at 11 hours post infection, both THC-11 and H-ras cell lines exhibited significant levels of HSV-1 protein synthesis, whereas cell line A549 did not exhibit detectable amounts of protein until 17 hours post infection. The Panel “B” of FIG. 9 shows that infection of H-ras cells by HSV-1 can be detected using immunofluorescence with anti-gC antibody, as early as 8 hours post-infection. Therefore, the use of transformed cell lines such as THC-1 I or H-ras can significantly shorten the time for the diagnosis of herpes virus infections in clinical samples. This would be important in situations where life-threatening herpes virus infections require immediate medical attention.

### EXAMPLE 3

The use of Farnesyl-Transferase Inhibitors to Reduce HSV-1Infection EfficiencyPost-translational farnesylation of Ras is necessary for association of Ras with the plasma membrane, and is a crucial process for the initiation of downstream events, including the three distinct MAPK cascades. It was postulated that if Ras, or downstream events initiated by Ras, is in fact involved in HSV-1 infection, then farnesyl transferase inhibitors should block HSV-1 replication in oncogene-transformed cells.

H-ras transformed NIH-3T3 cells were exposed to the farnesyl transferase inhibitors FTI-1 at a final concentration 50 μM or 100 μM in the culture medium. Control cells were not exposed to inhibitor. At 22 hours post-infection the cells were harvested and Western blot analysis was performed on these samples using rabbit anti-HSV-1 antibody, as described above. FIG. 4, panel “A” shows that compared to the control cells, which were not exposed to FTI-1 the production of viral proteins in cells that were exposed to FTI-1 is drastically reduced.

The bottom part of FIG. 4, Panel “B” shows the results of Western blots of identical samples as described in the paragraph above, except that they were probed with mouse anti-ERK1/2 antibody [Erk1/2] or mouse anti-phosphoERK1/2 antibody [P-Erk1/2]. These results show that, as a result of exposure to FTI-1, there is a lack of activity of MEK1/2 which leads to reduced phosphorylation of ERK1/2 (ERK42/44).

To determine whether other inhibitors of farnesyl transferase would have the same effect on viral protein synthesis, three additional inhibitors were tested. H-ras transformed NIH-3T3 cells were exposed to the farnesyl transferase inhibitors FPTI-1, FPTI-2 and FTI-4 at a final concentration 150 μM, 150 μM or or 50 μM in the culture medium. Control cells were not exposed to inhibitor. At 22 hours postinfection the cells were harvested and Western blot analysis was performed on these samples using rabbit anti-HSV-1 antibody, as described above. FIG. 5, panel “A” shows that compared to the control cells, viral protein synthesis in cells that were exposed to FPTI inhibitors is drastically reduced.

Panel “B” of FIG. 5 shows the effect of FTI-1 on HSV-1 infection, using immunofluorescence. As can be seen, addition of FTI-1 to the culture medium dramatically reduces the level of viral proteins detected and it appears that fewer cells are actually infected by HSV-1.

FIG. 6 shows the effects of FTI-1 at two different concentrations, on the virus yield, as quantitated by plaque titration on Vero cells (described above). Cells were infected at an MOI of 0.5 PFU/cell and were harvested 22 hours post infection. The inhibitors were present for the entire duration of the infection. As can be seen, FTI-1 treatment of cells dramatically decreased virus yield.

### EXAMPLE 4

The use of Inhibitors of the ERK Pathway to Reduce HSV-1 Infection EfficiencyA major pathway downstream of Ras that regulates cell growth is the ERK pathway [6]. Stimulation of this pathway requires the phosphorylation of ERK1/2 by the mitogen-activated extracellular signal-regulated kinase kinase MEK1/2 which itself is activated (phosphorylated) by Raf, a serine-threonine kinase downstream of Ras. To determine if MEK1/2 activity is required for HSV-1 infection, we studied the effect of the MEK1/2 inhibitor PD98059[10,16] on infected H-ras-transformed cells.

HSV-1 infected H-ras transformed NIH-3T3 cells were exposed to 40 μM PD98059 in the culture medium. Control cells (HSV-1 infected) were not exposed to PD98059. At 22 hours post-infection, the cells were harvested and Western blot analysis was performed on these samples using rabbit anti-HSV antibody, as described above. FIG. 4, panel “A” shows that compared to the control cells, which were not exposed to PD98059, the production of viral proteins in cells that were exposed to PD98059 is reduced.

FIG. 4 Panel “B” shows the results of Western blots of identical samples as described in the paragraph above, except that they were probed with mouse antiphosphoERK1/2 antibody [P-Erk1/2] to demonstrate that, as a result of exposure to PD98059, there is a lack of phosphorylation of ERK1/2 (Erk42/44).

As seen in FIG. 4, Panel “A”, the p38 kinase specific inhibitor, SB203580 had no effect on HSV-1 infection when used at effective doses, suggesting that elements downstream of p38 kinase are likely not involved with HSV-1 infection.

Panel “B” shows the results of Western blots of identical samples except that they were probed with mouse anti-phospho-ATF-2 antibody [P-ATF2]. These results show that, as a result of exposure to SB203580, there is a lack of phosphorylation of ATF-2.

FIG. 6 shows the effects of PD98059 on the virus yield, as quantitated by plaque titration on Vero cells (described above). Cells were infected at an MOI of 0.5 PFU/cell and were harvested 22 hours post infection. The inhibitor was present for the entire duration of the infection. As can be seen, PD98059 treatment of cells dramatically decreased virus yield, equivalent to the effect of FTI-1 on viral yield.

FIG. 6 also demonstrates the effects of SB203580 at two different concentrations, on the virus yield, as quantitated by plaque titration on Vero cells (described above). Cells were infected at an MOI of 0.5 PFU/cell and were harvested 22 hours post infection. The inhibitors were present for the entire duration of the infection. As can be seen, SB203580 treatment of cells does not effect virus yield

### EXAMPLE 5

P-13 Kinase Pathway is Not Involved in HSV-1 InfectionH-ras cells that were infected with HSV-1 were exposed to Wortmannin at a final concentration of 200 nM for the entire duration of the infection. Cells were harvested at 22 hr. post infection, lysed and subjected to SDS PAGE followed by immunoblotting with a rabbit anti-HSV-1 antibody. As the left panel of FIG. 7 shows, Wortmannin has no effect on HSV-1 infection. The bottom part of Panel “A” shows the results of Western blots of identical samples, except that they were probed with mouse antiphospho-Akt antibody [P-Akt]. The inhibitory effect of Wortmannin at this concentration is indicated by the lack of Akt phosphorylation.

The right panel of FIG. 7 shows the ability of HSV-1 to infect NIH-3T3 cells that express Ras effector domain mutants. The mutant cell lines V12C40, V12G37 and V12S35 all have a common activating G12V mutation as well as one other unique mutation in Ras, causing them to activate distinct pathways downstream of Ras. Mutant V12G37 is unable to signal via the RAF/ERK and the P13-kinase pathway, but allows signaling via the RAL-GDS pathway. The V12C40 mutation disrupts signaling via the RAF/ERK and the RAL-GDS pathways, but does not affect the P13-kinase pathway. The V12S35 mutant cannot signal through the RAL-GDS and the P13-kinase pathway, but can do so via the RAF/ERK pathway. Panel B of FIG. 7 shows that the V12S35 mutant is considerably more permissive to HSV-1 infection than the other two mutants, suggesting a more significant role of the RAF/ERK pathway (as compared with the P13-kinase or the RAL-GDS pathway) in this process.

### EXAMPLE 6

Viral Transcripts are Generated but not Translated in NIH-3T3 Cells To elucidate the role of the Ras pathway in HSV-1 infection, it was important to identify the step at which HSV-1 infection is blocked in NIH-3T3 cells. Virus binding and internalization is known to be comparable between permissive and non-permissive cells. Therefore, the transcription of viral genes was investigated. Functional protein products of the immediate early viral α genes are required for the subsequent transcription of the polypeptide groups β and γ.

The relative amounts of HSV-1 transcripts generated in HSV-1 infected NIH3T3 cells and H-ras-transformed cells was compared. Cells were infected with HSV-1 at a MOI of 0.5 PFU/cell. At 2, 5, 10, 20 and 25 hours after exposure to the virus, cells were harvested and RNA was extracted from them. Equal amounts of RNA from each sample were then subjected to RT-PCR, whereby selective amplification of specific viral cDNAs (α 27, U_{L}29, U_{L}30, γ₁34.5, and U_{L}44) was accomplished using the methods described in the “General Methods”. GAPDH, which is constitutively expressed, served as a PCR and gel loading control. FIG. 8, panel “A” shows that the immediate early transcript α 27 accumulated to comparable levels in the two cell lines. However, the β and γ transcripts were preferentially synthesized in the Ras-transformed cells and barely detectable, if at all, in the non-permissive NIH-3T3 cells.

Since transcription of these β and γ genes requires immediate early α gene products, the drastic reduction in their expression in NIH-3T3 cells was likely due to the inability of the α transcripts to be efficiently translated in these cells. Therefore, the level of the protein product of the α 27 gene (ICP27), the U_{L}29 gene (ICP8) and the U_{L}44 gene (gC) were compared between infected NIH-3T3 and H-ras -transformed cells. Cells at 20 hours post infection were harvested, and subjected to Western blot analysis using mouse anti-ICP27 antibody, as described in “General Methods”. FIG. 8, panel “B” shows that ICP27 was present at a much lower level in NIH-3T3 cells than in H-ras-transformed cells, even though the levels of α 27 transcripts were comparable between the two cell lines. Therefore, it appears that the α transcripts were not efficiently translated in NIH-3T3 cells, which in turn led to the lack of progression of downstream events as evidenced by the drastically reduced (or undetectable) levels of both the β and γ transcripts. Inefficient translation of these transcripts further reduced the output of the protein products such as gC and ICP8 to undetectable levels in NIH-3T3 cells as compared to H-rastransformed cells.

### EXAMPLE 7

PKR is Phosphorylated in HSV-1 Treated NIH-3T3 Cells, but not in HSV-1 Infected Oncogene-Transformed Cells.

Because viral transcripts were generated but not translated in NIH-3T3 cells, it was investigated whether PKR is activated (phosphorylated) in these cells. Phosphorylation of PKR leads to inhibition of translation of viral genes presumably because activated PKR phosphorylates the translation initiation factor eIF-2 α, which then inhibits translation of viral transcripts. In oncogene-transformed cells, PKR phosphorylation is prevented or reversed by Ras or one of its downstream elements, allowing viral gene translation to ensue.

NIH-3T3 cells and oncogene-transformed cells were infected with HSV-1 at a MOI of 0.5 PFU/cell, and incubated for 20 hours. Control (uninfected) cells were incubated for the same length of time. At this time, the media was aspirated off and the cells were lysed in a solution of: 20 mM HEPES, pH 7.4,120 mM KCI, 5 mM MgCl₂, 1 mM DTT, 0.5% Nonidet P-40, 2 μg/ml leupeptin, and 50 μg/ml aprotinin. The nuclei were then removed by low-speed centrifugation and the supernatants were stored at −70° C. until use.

Cytoplasmic extracts were normalized for total protein concentration, using the Bio-Rad protein microassay method. Each in vitro kinase reaction contained 20 μl of cell extract, 7.5 μl of reaction buffer (20 mM HEPES, pH 7.4, 120 mM KCI, 5 mM MgCl₂, 1 mM DTT and 10% glycerol) and 7.5 μl of ATP mixture (1.0 μCi [γ-³² P]ATP in 7 μl of reaction buffer), and was incubated for 30 minutes at 37° C. [25]. Aliquots were then either boiled in Laemmli SDS-sample buffer and used for Western Blotting with mouse monoclonal anti-PKR antibody for the detection of total PKR or immunoprecipitated with the same antibody followed by SDS-PAGE and autoradiography for the detection of ³² P-labelled PKR (FIG. 10). The phosphorylation state of eIF-2 α, the main substrate of PKR was also measured by Western blots using antibodies against total and phosphorylated forms of eIF-2 α.

The results of the Western Blot showed that PKR levels were comparable in all the four cell lines, whether infected with HSV-1, or not (FIG. 10, Panel “A”). However, PKR phosphorylation was seen only in infected cells, and was consistently more pronounced in NIH-3T3 cells than in the oncogene-transformed cells. PKR phosphorylation did not occur in uninfected NIH-3T3 cells, but did occur in infected NIH-3T3 cells, which suggests that it was a virus-triggered event (compare lanes 1 and 5). The differential phosphorylation of PKR between untransformed and transformed cells is consistent with the observed difference in their capacity to promote HSV-1 protein synthesis. As shown in the bottom of FIG. 10, Panel A, although the levels of eIF-2 α are constant, eIF-2 α phosphorylation is enhanced in NIH-3T3 cells upon HSV-1 infection, but not in transformed (HSV-1 sensitive) cells. The different phosphorylation of PKR and elF2 α between untransformed and transformed cells is consistent with the observed difference in their capacity to promote HSV-1 protein synthesis.

If inhibition of PKR phosphorylation is due to elements of the Ras signaling pathway then FTI-1 and PD98059 (blockers of Ras plasma membrane association and MEK activity respectively) which effectively inhibit HSV-1 protein synthesis and HSV-1 virus production in transformed cells, should restore PKR phosphorylation in infected cells. FIG. 10 Panel “B” shows the effect of FTI-1 and PD98059 on PKR phosphorylation in HSV-1 infected H-ras cells. H-ras cells were exposed to FTI-1 (100 μM) or PD98059(40 μM) and the phosphorylation state of PKR was assessed using anti-PKR (total PKR) or anti-phospho-PKR (P-PKR) antibodies . As can be seen, PKR phosphorylation is restored by these inhibitors. The difference in phosphorylation of PKR in infected NIH-3T3 cells and in infected MEF cells is shown in the right side of FIG. 10, Panel “B”. As can be seen, MEF cells, which are sensitive to HSV-1, have much lower levels of phosphorylated PKR than do NIH3T3 cells, which are relatively non-permissive to HSV-1 infection.

### EXAMPLE 8

Oncogene-Transformed Cells are more Permissive to HSV-1 Mutant R3616 than are NIH-3T3 Cells The permissiveness of oncogene-transformed NIH-3T3 cell lines to attenuated mutants of HSV-1 was assessed by Western blot analysis. NIH-3T3 cell lines, and oncogene-transformed cell lines were infected with Mutant R3616 [17, 22-24]. Cells were harvested at 20 hours after infection and subjected to Western blotting with rabbit anti-HSV-1 antibody, as described in the “General Methods”. As shown in FIG. 11, as evidenced by the fact that they make substantially more viral proteins, oncogene-transformed cell lines are more permissive to R3616 than NIH-3T3 cells.

### EXAMPLE 9

PKR Deletion Enhances the Permissiveness of Cells to HSV-1 Infection Mutant R3616 contains deletions in both copies of the γ₁34.5 gene. The gene product of γ₁34.5 (called ICP34.5) presumably forms a complex with protein phosphatase 1 and redirects its activity to dephosphorylate eIF-2 α. Once dephosphorylated, eIF-2 α is inactivated and unable to inhibit viral transcription. The net result of iCP34.5 activity is to dephosphorylate eIF-2 α, whereas the activity of phosphorylated PKR is to phosphorylate eIF-2 α. Thus, ICP34.5 plays an antagonistic role to PKR-i.e. it acts “anti-PKR”, and mutant R3616 can be regarded as a virus that has lost its inherent anti-PKR mechanism.

A direct approach to test these hypotheses, and to define the role of PKR in HSV-1 infection, is through the use of host cells that are devoid of the PKR gene. Host cells that have lost the PKR gene should be more permissive to R3616 infection than host cells that have the PKR gene. In the absence of PKR, the absence of an anti-PKR mechanism in mutant R3616 is of no consequence. Conversely, wild-type HSV-1 infectivity should not differ between host-cells that do or do not have PKR activity. Since the anti-PKR activity is working in wild-type HSV-1, the presence of PKR in the host cell is of negated by the viral anti-PKR activity.

Primary embryo fibroblasts from PKR⁻/⁻ mice and PKR⁺/⁺ mice were compared in terms of permissiveness to HSV-1 and R3616 infection. Cells were infected with MOI of 0.5 PFU/cell and 20 hours after infection were harvested and subjected to Western blotting with rabbit anti-HSV-1 antibody, as described in the “General Methods”. FIG. 12 (right two lanes) shows that wild-type HSV-1, armed with its own anti-PKR mechanism, was able to infect PKR⁻/⁻ and PKR⁺/⁺ mouse embryo fibroblasts equally well. In contrast, R3616 viral proteins were synthesized at a significantly higher level in the PKR⁻/⁻ cells than in the PKR⁺/⁺ cells (FIG. 12, left two lanes).

The level of infection by R3616 in PKR⁻/⁻ fibroblasts is equivalent to that seen in cells transformed by Ras, or elements of the Ras pathway. Therefore, PKR deletion enhances host cell permissiveness to HSV-1 infection in the same way as does transformation by ras or elements of the Ras pathway. Further evidence for the Ras-PKR connection comes from the demonstration that FTI-1 inhibited HSV-1 infection of PKR⁺/⁺ cells, while having no effect on PKR⁻/⁻ cells, as shown in FIG. 13. In this experiment, cells were infected with HSV-1 at a MOI of 0.5 PFU/cell, in the presence of FTI-1. Cells were harvested at 20 hr post-infection and analyzed for viral proteins as above. Viral protein synthesis was reduced by FTI-1 in only the PKR⁺/⁺ cells.

REFERENCES The following references are cited in the application as numbers in brackets ([ ]) at the relevant portion of the application. Each of these references is incorporated herein by reference.

1. Whitley et al., “Herpes simplex viruses” Clin. Infect Dis. 26: 541-553 (1998).

2. Subak-Sharpe and Dargan “HSV Molecular Biology: General Aspects of Herpes Simplex Virus Molecular Biology” Virus Genes 16: 3, 239-251 (1998).

3. Whitmarsh et al., “Integration of MAP kinase signal transduction pathways at the serum response element” Science 269: 403-407 (1995).

4. Gibbs etal., “Farnesyl transferase inhibitors versus Ras inhibitors” Curr. Opin. Chem. Biol. 1: 197-203 (1997).

5. Heimbrook and Oliff “Therapeutic intervention and signaling” Curr. Opin. Cell Biol. 10: 284-288 (1998).

6. Robinson and Cobb, “Mitogen-activated protein kinase pathways” Curr. Opin. Cell Biol. 9: 180-186 (1997).

7. Shields et al. , “Understanding Ras: “it ain”t over “til it”s over”” Trends Cell Biol. 10: 147-154 (2000)

8. Garrington and Johnson “Organization and regulation of mitogen-activated protein kinase signaling pathways” Curr. Opin. Cell Biol. 11: 211-218 (1999).

9. Roovers and Assoian, “Integrating the MAP kinase signal into the G1 phase cell cycle machinery” Bioessays, 22: 818-826 (2000).

10. Dudley et al., “A synthetic inhibitor of the mitogen-activated protein kinase cascade. Proc. Natl. Acad. Sci. USA 92: 7686-7689 (1995).

11. Cuenda et al., “SB 203580 is a specific inhibitor of a MAP kinase homologue which is stimulated by cellular stresses and interleukin-1” F. E.B.S. Lett. 364: 229-233 (1995).

12. Nakamura et al., “Phosphatidylinoditol-3 kinase is involved in ruffled border formation in osteoclasts” J. Cell. Physiol. 172: 230-239 (1997).

13. White et. al., “Multiple Ras functions can contribute to mammalian cell transformation” Cell 24: 533-541 (1995).

14. Khosrave-Far et. al. , “Oncogenic Ras activation of Raf/mitogen-activated protein kinase-independent pathways is sufficient to cause tumorigenic transformation” Mol. Cell. Biol. 16: 3923-3933 (1996).

15. Webb et al., “Signaling pathways in Ras-mediated tumorigenicity and metastasis” Proc. Natl. Acad. Sci. U.S.A. 21: 8773-8778 (1998).

16. Coffey et al., “Reovirus Therapy of Tumors with Activated Ras Pathway” Science 282: 1332-1334 (1998).

17. Chambers etal., “Comparison of genetically engineered herpes simplex viruses for the treatment of brain tumors in a scid mouse model of human malignant glioma” Proc. Natl. Acad. Sci. USA 92: 1411-1415 (1995).

18. Roizman and Sears in Fundamental Virology(eds. Fields, Knipe and Howley) pp 1403 1107 (Lippencott-Raven, New York, 1996).

19. “The γ₁34.5 protein of herpes simplex virus 1 α complexes with protein phosphatase 1 α to dephosphorylate the α subunit of the eukaryotic translation initiation factor 2 and preclude the shutoff of protein synthesis by double-stranded RNA-activated protein kinase” Proc. Natl. Acad. Sci. USA 94: 843-848 (1997).

20. Williams et al, “PKR; a sentinel kinase for cellular stress” Oncogene 18: 6112-6120 (1999)

21. Yang et al., EMBO J. 14:6095-6106 (1995); Der etal, Proc.

Natl. Acad. Sci. USA, 94:3279-3283 (1997)).

22. Andreansky etal., “The application of genetically engineered herpes simplex viruses to the treatment of experimental brain tumors” Proc. Natl. Acad. Sci. U.S.A. 93: 11313-11318 (1996).

23. Andreansky et al., “Evaluation of genetically engineered herpes simplex viruses as oncolytic agents for human malignant brain tumors” Cancer Res. 57: 1502-1509 (1997).

24. Chou et al., “Mapping of herpes simplex virus-1 neurovirulence to gamma 134.5, a gene nonessential for growth in culture” Science 250: 1262-1266 (1990).

25. Mundschau and Faller “Platelet-derived growth factor signal trnasduction through the interferon-inducible kinase PKR. Immediate early gene induction” J. Biol. Chem., 267: 23092-23098 (1992)

26. Farassati, et al. “Oncogenes in Ras Signaling Pathway Dictate Host Cell Permissiveness to Herpes Simplex Virus 1” Nature Cell Biology (in press, 2001)

1. A method of treating a viral infection in a mammal comprising administering to cells of said mammal that are infected with a virus which utilizes the host cell Ras pathway or otherwise exploits a host cell anti-PKR mechanism, in sufficient amount that viral replication in infected cells is inhibited, an agent selected from a group comprising:
(a) agents that inhibit Ras pathway function or activity;
(b) agents that promote, allow or increase PKR phosphorylation, function or activity; or
(c) agents that promote, allow or increase eIF-2 α phosphorylation, function or activity.

2. The method of claim 1 wherein the virus is a herpes virus.

3. The method of claim 1 wherein the agent interferes with the ability of Ras to associate with the plasma membrane.

4. The method of claim 1 wherein the agent interferes with the post-translational farnesylation of Ras.

5. The method of claim 1 wherein the agent is a farnesyl transferase inhibitor or a farnesyl protein transferase inhibitor.

6. The method of claim 5 wherein the farnesyl transferase inhibitor is selected from a group comprising FTase Inhibitor I, FTase Inhibitor II, FTase Inhibitor III, FTase Inhibitor IV, FTI 276, FTI 277, FPT Inhibitor I, FPT Inhibitor II and FPT Inhibitor III.

7. The method of claim 1 wherein the agent is an ERK pathway inhibitor.

8. The method of claim 1 wherein the agent is a MEK1/2 inhibitor.

9. The method of claim 8 wherein the MEK1/2 inhibitor is PD98059, U0124, U0125 or U0126.

10. The method of claim 1 wherein the agent is an agent that inhibits Ras pathway function or activity.

11. The method of claim 1 wherein the agent is an agent that promotes, allows or increases PKR phosphorylation, function or activity.

12. The method of claim 1 wherein the agent is an agent that promotes, allows or increases eIF-2 α phosphorylation, function or activity.

13. The method of claim 2 wherein the herpes infection is a HSV-1 or HSV-2 infection.

14. The method of claim 1 wherein the mammal is a human.

15. A pharmaceutical composition for treating a mammalian viral infection comprising an agent and a pharmaceutically acceptable excipient, wherein the agent is selected from a group comprising:
(a) agents that inhibit Ras pathway function or activity;
(b) agents that promote, allow or increase PKR phosphorylation, function or activity; and
(c) agents that promote, allow or increase eIF-2 α phosphorylation, function or activity.

16. The composition of claim 15 wherein the viral infection is a herpes infection.

17. The composition of claim 15 wherein the agent interferes with the ability of Ras to associate with the plasma membrane.

18. The composition of claim 15 wherein the agent interferes with the post-translational farnesylation of Ras.

19. The composition of claim 15 wherein the agent is a farnesyl transferase inhibitor or a farnesyl protein transferase inhibitor.

20. The composition of claim 19 wherein the farnesyl transferase inhibitor is selected from a group comprising FTase Inhibitor I, FTase Inhibitor II, FTase Inhibitor III, FTase Inhibitor IV, FTI 276, FTI 277, FPT Inhibitor I, FPT Inhibitor II and FPT Inhibitor III, FPT Inhibitor IV.

21. The composition of claim 15 wherein the agent is an ERK pathway inhibitor.

22. The composition of claim 15 wherein the agent is a MEK1/2 inhibitor.

23. The composition of claim 22 wherein the MEK1/2 inhibitor is PD98059, U0124, U0125 or U0126.

24. The composition of claim 15 comprising an agent which inhibits Ras pathway function or activity.

25. The composition of claim 15 comprising an agent which promotes, allows or increases PKR phosphorylation, function or activity.

26. The composition of claim 15 comprising an agent which promotes, allows or increases eIF-2 α phosphorylation, function or activity.

27. The composition of claim 16 wherein the herpes infection is a HSV-1 or HSV-2 infection.

28. The composition of claim 15 wherein the mammal is a human.

29. A method of preventing a viral infection in a mammal comprising administering to cells of said mammal, in sufficient amount that viral replication in the cells is prevented, an agent selected from a group comprising:
(a) agents that inhibit Ras pathway function or activity;
(b) agents that promote, allow or increase PKR phosphorylation, function or activity; and
(c) agents that promote, allow or increase eIF-2 α phosphorylation, function or activity.

30. A method of diagnosing a herpes infection in a mammal comprising the steps of:
(a) providing cultured mammalian cells with an activated Ras pathway;
(b) inoculating the cells with a specimen from the mammal;
(c) incubating the cells for a length of time sufficient to allow viral protein synthesis to proceed within the cells; and
(d) detecting viral proteins that are synthesized by the cells.

31. The method of claim 30 wherein the cells are selected from the group comprising NIH-3T3, NR6 and Swiss 3T3 cells.

32. The method of claim 30 wherein the cells have been generated by transfection with an oncogene.

33. The method of claim 32 wherein the oncogene is selected from the group comprising v-erbB, activated Sos and activated ras.

34. The method of claim 30 wherein the viral proteins are detected using antibodies directed against the viral proteins.

35. The method of claim 34 wherein the antibodies detect viral α proteins, viral β proteins or viral γ proteins.

36. The method of claim 35 wherein the α protein is ICP27, ICP4 or ICP0.

37. The method of claim 35 wherein the β protein is ICP8.

38. The method of claim 30 wherein the viral proteins are detected by immunofluorescence or immunoblotting.

39. The method of claim 30 wherein the herpes infection is a HSV-1 or HSV-2 infection.

40. The method of claim 30 wherein the mammal is a human.

41. A kit for diagnosing herpes infections, which kit comprises:
(a) cultured mammalian cells that have an activated Ras pathway; and
(b) means of detecting viral proteins that are synthesized by the cells.

42. The kit of claim 41 wherein the means for detecting viral proteins comprises an antibody to a viral protein and a means for detecting the anti body.

43. The kit of claim 41 wherein the means for detecting the antibody is a fluorescent body coupled to the antibody.

44. The kit of claim 41 wherein the cultured mammalian cells are transfected with a gene which activates the Ras pathway.

45. The kit of claim 44 wherein the cultured cells are selected from the group consisting of NIH-3T3, NR6 and Swiss 3T3 cells.

46. The kit of claim 45 wherein the gene is an oncogene.

47. The kit of claim 46 wherein the oncogene is selected from the group consisting of v-erbB, Sos and H-ras.
